(19) United States
(12) Patent Application Publication
(10) Pub. No.: US 2007/0092588 A1
(43) Pub. Date: Apr. 26, 2007

(54) Soy milk made from germinated soy beans

(75) Inventors: Akira Nagatoishi, Yokosuka (JP); Haruna Aida, Yokosuka (JP)

Correspondence Address: David B. Ritchie;Thelen Reid & Priest, LLP, P.O. Box 640640, San Jose, CA, 95164-0640, US

(73) Assignee: The Nisshin OilliO Group, Inc.

(21) Appl. No.: 11/431,224

(22) Filed: May 9, 2006

(30) Foreign Application Priority Data
Oct. 21, 2005 (JP) 2005-306724

(51) Int. Cl. A61K 36/48

(52) U.S. Cl. 424/757

(57) ABSTRACT

Soymilk is provided which can prevent or alleviate menopausal disorders, and is highly safe with no risk of side effects even with long-term intake thereof. This soymilk is made from germinated soybeans, and is used for alleviating or preventing the vasomotor disorder-like symptoms and psychoneuropathic disorder-like symptoms associated with menopausal disorders. Furthermore, this soymilk is labeled with a notice specifying that the soymilk is to be used for alleviating or preventing the vasomotor disorder-like symptoms and psychoneuropathic disorder-like symptoms of menopausal disorders.

This application is based on and claims the benefit of priority from Japanese Patent Application No. 2005-306724, filed on 21, Oct. 2005, the content of which is incorporated herein by reference.

### BACKGROUND OF THE INVENTION

1. Field of the Invention

The present invention relates to soymilk prepared using germinated soybeans as a main ingredient (germinated soybean milk), which may be used for alleviating symptoms of menopausal disorders.

2. Related Art

Menopausal disorders cover various symptoms caused by hormone imbalances due to decreases in estrogen in women who have reached menopause, continuing for several years until the end thereof. Menopausal disorders are caused by hypoovarianism, environmental factors, psychogenic factors, or the like, and are mainly classified as symptoms like those of vasomotor disorders, psychoneuropathic disorders, dysesthetic disorders, urogenital system disorders, locomotor disorders, dermal and urinary disorders, digestive system disorders, and the like. For these symptoms, hormone-replacement therapy has been performed to supplement hormones such as estrogen. However, while a dramatic improvement in symptoms can often be achieved by the hormone-replacement therapy in spite of the presence of multiple symptoms, there is a risk of carcinogeneis due to side effects of hormones, leading to breast cancer, uterine cancer, etc. Furthermore, some menopausal disorders are estrogen-independent so that no improvement can be expected by the hormone treatment in patients with such disorders, and moreover, a very large number of patients complain of vague symptoms which are difficult to objectively diagnose.

Menopause is a period in which women experience various symptoms, causing mental distress regardless of the severity thereof. Under these circumstances, there has been a constant demand to improve the quality of life (QOL) of women by preventing or alleviating menopausal symptoms or disorders.

In this regard, isoflavones are flavonoids contained mainly in soybeans, which act like female hormones, and their use in alleviating various symptoms caused by female hormone imbalances such as menopausal disorders, osteoporosis, hyperlipemia, and obesity has been disclosed (Patent document 1: Japanese Unexamined Patent Application, First Publication No. 2003-2831).

Additionally, γ-aminobutyric acid (hereinafter, referred to as GABA) is an amino acid biosynthesized from glutamic acid, which exists mainly in the brains of animals, acting as a principal inhibitory neurotransmitter, and its superior actions in preventing and alleviating symptoms of menopausal disorders have been reported (Non-patent document 1: Nippon Shokuhin Kagaku Kogaku Kaishi, August 2000, Vol. 47, No. 8, pp. 593-603).

Furthermore, there has been disclosed a preventive and symptom-alleviating agent for menopausal disorders containing γ-aminobutyric acid and isoflavones in amounts that prevent or alleviate symptoms of menopausal disorders (Patent document 2: Japanese Unexamined Patent Application, First Publication No. 2005-139135).

Beans have been used as food from long ago because of their excellent nutritive value in carbohydrates, protein, fat, etc. Among them, soybeans in particular are referred to as the “meat from the fields,” because they are rich in protein and also yield a large quantity of soybean oil that contains essential fatty acids. Furthermore, recently, it has been scientifically demonstrated that soybean protein lowers the risk of cardiac disease and reduces cholesterol levels. Also, soybean isoflavones, as another nutrient, have been studied for protective action against osteoporosis. Soybean-processed foods containing soybeans as their main ingredient have existed in the Japanese diet from centuries ago as a traditional food. These include fermented soybean paste, fermented soybean, bean curd, soymilk, etc. On the other hand, soymilk has recently expanded its market not only due to strong interest in diet and health but also due to the threat of mad cow disease so that various soymilk-based drinks have been. actively developed.

### SUMMARY OF THE INVENTION

However, preventive or alleviating effects of soybean isoflavones on menopausal disorders according to Patent Document 1 cannot be accepted as being sufficient. Furthermore, Patent Document 2 discloses the discovery of effectiveness of γ-aminobutyric acid in preventing or alleviating menopausal disorders and the use thereof together with isoflavones. However, it used γ-aminobutyric acid obtained by glutamic acid fermentation with lactic acid bacillus, but did not investigate the use of soymilk prepared using germinated soybeans (germinated soybean milk) as a main ingredient in alleviating or preventing menopausal disorders. Furthermore, using defatted rice germ of γ-aminobutyric acid accumulation, Non-patent Document 1 describes research concerning the fact that the intake of γ-aminobutyric acid, the natural intake of which from conventional foods is quantitatively limited, can be elevated to levels at which physiological function can be expected. However, it did not also disclose the utilization of soymilk made from germinated soybeans in alleviating or preventing menopausal disorders.

Thus, although the use of γ-aminobutyric acid and isoflavones in alleviating or preventing menopausal disorders has been developed, no studies have been made on enrichment of γ-aminobutyric acid in soybeans by germinating them and on utilization of soymilk made from germinated soybean in alleviating or preventing menopausal disorders.

In view of the above-described problems, an object of the present invention is to provide soymilk that is not only usable for preventing or alleviating menopausal disorders but is also highly safe even for long-term consumption.

The inventors of the present invention have diligently carried out researched to solve the above-described problems, and as a result, have discovered that soymilk prepared using soybeans which have been subjected to the germination process to enrich their γ-aminobutyric acid content is capable of efficiently alleviating or preventing menopausal disorders by giving the soymilk to subjects, preferably women with symptoms caused by menopausal disorders, thereby achieving the invention.

More specifically, the present invention provides the following.

In a first aspect of the present invention, soymilk is made from germinated soybeans, and is usable in alleviating or preventing at least one of vasomotor disorder-like symptoms and psychoneuropathic disorder-like symptoms associated with menopausal disorders.

In a second aspect of the present invention, soymilk is made from germinated soybeans according to the first aspect of the present invention, wherein said vasomotor disorder-like symptoms are one or more symptoms selected from a group comprising heat, poor circulation, hot flashes, palpitation, tachycardia, and bradycardia, and wherein said psychoneuropathic disorder-like symptoms are one or more symptoms selected from a group comprising headache, vertigo, insomnia, susurrus aurium, phobias, oppressive feelings, and hypomnesia.

In a third aspect of the present invention, soymilk is made from germinated soybeans according to the first aspect of the present invention, wherein said soymilk is to be used for long-term improvement of the quality of life (QOL) of people experiencing menopausal disorders.

In a fourth aspect of the present invention, soymilk is made from germinated soybeans according to any one of the first through third aspects of the present invention, wherein the soymilk contains γ-aminobutyric acid and isoflavones, and wherein the content of the γ-aminobutyric acid in 100 mL of the soymilk is not less than 8 mg and that of the isoflavones in 100 mL of the soymilk is not less than 15 mg.

In a fifth aspect of the present invention, soymilk is made from germinated soybeans according to the forth aspect of the present invention, wherein the soymilk contains serine, methionine, and phenylalanine, and has a solid content of not less than 8%, and wherein the content of said serine per 100 mL of the soymilk is not less than 2 mg, that of said methionine per 100 mL of the soymilk is not less than 1 mg, and that of said phenylalanine per 100 mL of the soymilk is not less than 2 mg.

The above-described soymilk made from germinated soybeans according to the present invention is capable of preventing or alleviating a variety of physical and mental symptoms associated with menopausal disorders and is also highly safe even for long-term intake thereof because of no risk of side effects.

That is, the soymilk of the present invention made from soybeans that have been subjected to a germination process so as to enrich γ-aminobutyric acid (in the present application, this may be referred to as germination-processed soybean milk (germinated soybean milk)) has a high γ-aminobutyric acid content and also contains the soybean isoflavones contained in soybeans. Therefore, this soymilk facilitates the daily intake of γ-aminobutyric acid and isoflavones in amounts required for preventing menopausal disorders and alleviating symptoms associated with menopause. Furthermore, soymilk also contains, in addition to γ-aminobutyric acid and isoflavones, other ingredients represented by various free amino acids. Hence, the present invention exhibits excellent effects in preventing menopausal disorders and alleviating symptoms associated therewith due to synergistic effects among these ingredients. Therefore, the present invention is extremely useful for improving the quality of life of women preferably over a long period of time both before and after menopause. Furthermore, the present invention has found that the soymilk of this invention is effective in alleviating or preventing at least one of vasomotor disorder-like symptoms and psychoneuropathic disorder-like symptoms in particular among various menopausal disorders.

Herein, the quality of life (QOL) refers to a feeling of satisfaction or a feeling of fulfillment experienced by a patient having a specific disorder, specifically the extent to which items (11) to (60) concerning discomfort in daily life, as described in the Examples below, are not experienced by the patient. That is, among these items concerning discomfort, the smaller the number of items that actually apply to a patient, the higher the quality of life (QOL) of the patient.

Furthermore, “improvement of QOL” indicates a decrease in the number of items concerning discomfort as in (11) to (60) in the Examples, as experienced by the patient as time goes by.

### DETAILED DESCRIPTION OF THE INVENTION

Hereinafter, preferred embodiments of soymilk of the present invention, prepared using germinated soybeans as its main ingredient, are specifically described.

In the present invention, soymilk made from germinated soybeans is prepared by carrying out germination processing as below, so as to process the germinated soybeans as its main ingredient.

First, germinated soybeans used in this invention are explained.

Germinated Soybeans

Germinated soybeans are soybeans which have been immersed in water so as to absorb the water required for the germination reaction, and after draining the water or during the immersion process, are exposed to air or oxygen to promote germination thereof while maintaining a desirable temperature and humidity, regardless of whether they are actually germinated or not. Specifically, for example, drained soybeans are transferred to a germination bed and intermittently sprayed with water, or wrapped with wet cloth to promote germination. A commonly used germination bed can be used as a germination apparatus in the present invention, although there is no limitation thereto.

The specific method of germinating soybeans is not particularly limited, and may include methods including an immersion process in which soybeans are immersed in water or warm water at 10 to 45 degrees Celsius, preferably 20 to 45 degrees Celsius, or more preferably 30 to 42 degrees Celsius, for 0.5 to 36 hours, preferably 1 to 10 hours, or more preferably 1 to 5 hours, and an air-contacting process in which soybeans are exposed to air or oxygen, for example, at 25 to 45 degrees Celsius, or more preferably 25 to 35 degrees Celsius for 19 to 36 hours, preferably 20 to 30 hours, or more preferably 20 to 24 hours, during or after the immersion process.

In the present invention, any variety of soybeans may be used, such as those grown in Japan, soybeans grown in the U.S. such as IOM, genetically modified soybeans, or non-genetically modified soybeans.

Germinated soybeans such as those described in International Patent Publication No. WO2005/004633 can be preferably used.

Preparation of Soymilk

Next, soymilk is prepared using the above-described germinated soybeans according to an ordinary method. Specifically, for example, soymilk is prepared by crushing the germinated soybeans obtained above while adding water thereto, separating the produced soybean curd pulp (lees), if necessary, and heat-sterilizing the separated liquid (liquid in which particles are further refined using a mixer or the like when soybean curd pulp is not separated) by an instantaneous heater that directly blows steam. Soymilk is prepared also by drying and crushing the above-described germinated soybeans to form whole-grain soybean flour, dissolving (suspending) it in water, and heat-sterilizing the resultant liquid. In these cases, soymilk may be flavored with sugars, fruit juices, and such, prior to heat-sterilization so as to make it more palatable. Thus, soymilk having excellent taste and high commercial value can be prepared.

Soymilk according to the present application refers to not only soymilk which meets the Japanese Agricultural Standard (JAS) but also all soymilk-like preparations containing beans as a main ingredient not limited to JAS, including, for example, those not separated from bean curd pulp (lees) and those prepared by drying beans, pulverizing them once, and dissolving the resulting flour in water.

Contents of γ-Aminobutyric Acid and Isoflavone

Soymilk according to the present invention prepared using the above-described germinated soybeans contains soybean solid content of not less than 4%, or preferably not less than 8%, and is enriched with γ-aminobutyric acid due to the germination process of soybeans. The γ-aminobutyric acid content is not particularly limited; however, the content in 100 mL of the soymilk is preferably not less than 8 mg, more preferably not less than 10 mg, and most preferably not less than 12 mg.

Herein, the γ-aminobutyric acid content in soymilk can be obtained by analysis using an automatic amino acid analyzer. Specifically, after the soymilk is mixed and stirred with 5% trichloroacetic acid, the mixture is centrifuged, and the supernatant is filtered using a filter to obtain a filtrate. The filtrate thus obtained is analyzed using an automatic amino acid analyzer.

Isoflavones are flavonoid compounds contained in plants in the genus Glycine of Leguminosae. Specifically, it refers to one or more types of compounds selected from a group including daidzin, daidzein, genistin, genistein, glycitin, glycitein, acetyldaidzin, acetylgenistin, acetylglycitin, malonyldaidzin, malonylgenistin, and malonylglcitin. It is generally known that these isoflavones are effective in preventing osteoporosis, menopausal disorders, etc. Although the isoflavone content is not particularly limited, soymilk according to the present invention made from the above-described germinated soybeans contains in 100 mL of the soymilk preferably not less than 15 mg, more preferably not less than 20 mg, and most preferably not less than 25 mg of this isoflavone. Herein, the isoflavone content (mg) can be obtained by analysis using a conventionally known HPLC (High-Performance Liquid Chromatography) method.

Other Ingredients

Furthermore, soybeans are enriched by the germination process with free amino acids such as serine, methionine, and phenylalanine in addition to the above-described γ-aminobutyric acid. Since these ingredients exert ataractic effects, they act effectively against psychoneuroses associated with menopausal disorders. Contents of serine, methionine, and phenylalanine are not particularly limited, but the soymilk according to the present invention made from the above-described germinated soybeans contains preferably 2 mg or more, more preferably 2.25 mg or more, and most preferably 2.5 mg or more serine per 100 mL of the soymilk. The aforementioned soymilk also contains preferably 1 mg or more, more preferably 1.15 mg or more, and most preferably 1.3 mg or more methionine per 100 mL of the soymilk. The aforementioned soymilk also contains preferably 2 mg or more, more preferably 2.25 mg or more, and most preferably 2.5 mg or more phenylalanine per 100 mL of the soymilk.

Effective Intake

Daily intake of γ-aminobutyric acid and isoflavones necessary for alleviating and preventing symptoms associated with menopausal disorders is not particularly limited, but the daily intake of γ-aminobutyric acid is usually in the range of 10 to 50 mg, and preferably 20 to 40 mg. Furthermore, the daily intake of soybean isoflavones is usually in the range of 20 to 150 mg as total isoflavones, and preferably 30 to 100 mg. It was demonstrated that daily intake of the above-described amounts of γ-aminobutyric acid and isoflavones is effective in alleviating and preventing symptoms of menopausal disorders.

The effective intake of soymilk made from germinated soybeans can be appropriately determined taking the condition of subjects, severity of symptoms to be prevented or alleviated, and the like, into consideration. Although the effective intake of soymilk is not particularly limited as long as the above-described daily intake is satisfied, the aforementioned soymilk is taken, for example, preferably in the range of 100 to 500 mL per day.

Menopausal Symptoms

Menopausal symptoms to be alleviated by soymilk or foods (processed foods prepared using the soymilk of the present invention and foods containing the above-described soymilk or processed foods) made from germinated soybeans include the vasomotor disorder-like symptoms such as heat, poor circulation, hot flashes, palpitation, tachycardia, and bradycardia; psychoneuropathic disorder-like symptoms such as headache, vertigo, insomnia, susurrus aurium, phobias, oppressive feelings, and hypomnesia; locomotor disorder symptoms such as stiff neck, lumbago, arthralgia, spondylalgia, myalgia, and sciatic ache; sensory disorder symptoms such as numbness, hyperesthesia, anesthesia, and formication; urogenital system disorder symptoms such as pollakiuria and dysuria; dermal and urinary system disorder symptoms such as sudoresis, xerostomia, and sialism; digestive system disorder symptoms such as anorexia, nausea, emesis, constipation, and diarrhea; as well as other symptoms such as fatigue (lassitude) and bellyache. Mitigation of such unpleasant symptoms of menopausal disorders enables women to comfortably go through menopause. Indicators of “comfortably going through menopause” may include, for example, the improvement of quality of life described below in the Examples. Furthermore, soymilk or foods of the present invention made from germinated soybeans are excellent in effectively alleviating or preventing the vasomotor disorder-like symptoms and psychoneuropathic disorder-like symptoms in particular, among the above-described menopausal disorders. Therefore, soymilk or foods of this invention can be used as a preventive and alleviative agent for preventing or alleviating the vasomotor disorder-like symptoms and psychoneuropathic disorder-like symptoms of menopausal disorders.

In this case, although not specifically shown in the following Examples, tachycardia and bradycardia which are symptoms of menopausal disorders classified into vasomotor disorder-like symptoms are caused by mechanisms similar to those of other vasomotor disorder-like symptoms described below so that similar symptom-alleviating effects are thought to be obtained for these symptoms.

Similarly, for susurrus aurium, phobias, oppressive feelings, and hypomnesia, which are classified into psychoneuropathic disorder-like symptoms, symptom-alleviating effects similar to those for other psychoneuropathic disorder-like symptoms are thought to be obtained.

In addition, the above-described soymilk or foods exhibit effects to improve quality of life by the mere intake thereof as described below in the Examples, so that they are extremely effective in improving the quality of daily life.

Notice

Since, in this invention, notices specifying that the soymilk is to be used for alleviating or preventing vasomotor disorder-like symptoms and psychoneuropathic disorder-like symptoms are notices that are permitted primarily for specified health foods. The notices may include those such as “will mitigate unpleasant menopausal symptoms”, “for those suffering from unpleasant symptoms before and after menopause”, “for comfortably going through menopause”, and “for those disturbed by menopausal hot flashes, perspiration, breathlessness or palpitation, insomnia, annoyance, depression and headache”.

Furthermore, the notice specifying that the article is to be used for alleviating unpleasant menopausal symptoms may include notices permitted primarily for foods for non-specified health use, such as “to cheer you up”, “to relieve hot flashes and feel refreshed”, “to make you feel nice and warm”, “to sleep soundly”, and “mind”.

This notice that specifies that the soymilk is to be used for alleviating unpleasant symptoms of menopausal disorders conveys a broader concept than the above-described notice specifying that the soymilk is to be used for alleviating or preventing the vasomotor disorder-like symptoms and psychoneuropathic disorder-like symptoms. Hence, in the present invention, the notice specifying that the soymilk is to be used for alleviating unpleasant symptoms of menopausal disorders may be written separately or jointly with the one specifying that the soymilk is to be used for alleviating or preventing the vasomotor disorder-like symptoms and psychoneuropathic disorder-like symptoms.

Moreover, these notices can be attached to container packaging by conventionally known methods, so as to clearly express that the soymilk of this invention prepared from germinated soybeans is to be used in alleviating or preventing psychoneuropathic disorder-like symptoms of menopausal disorders or mitigating unpleasant symptoms thereof. Thus, the soymilk of this invention is clearly distinguished from ordinary soymilk.

Processed Foods Prepared Using Soymilk of the Present Invention and Foods Containing the Above-Described Soymilk or Processed Foods

Since processed foods prepared using the above-described soymilk also contain almost all of the constituents of the soymilk, they exhibit similar effects to those of the soymilk. They include soybean curd, soybean curd pudding, etc. When the above-described soymilk or processed foods are contained in other foods in an amount sufficient for exhibiting the effects, these foods will naturally exhibit similar effects. The above-described soymilk or foods containing the above-described processed foods can-be used also in preparing non-bean-processed foods including bread, pizza, noodles such as udon, buckwheat noodles and somen noodles, dairy products such as ice cream, pudding and yogurt, and confectionary such as biscuits, rice crackers, sliced and dried rice cakes, cubed rice crackers, puddings, and Japanese cakes.

### EXAMPLES

Next, examples and comparative examples are given to describe the present invention in more detail. The present invention, however, is not limited to these examples.

### Example 1

### Preparation of Soymilk from Germinated Soybeans

After 300 kg of US-produced IOM non-germinated soybeans were immersed in 1000 L of warm water at a temperature of 30 degrees Celsius for 2 hours, germination was stimulated in air while spraying water at a temperature of 20 degrees Celsius every 6 hours for 24 hours to obtain 690 kg of germinated soybeans. After 690 kg of the germinated soybeans thus obtained were crushed while adding water thereto, the liquid obtained by separating soybean curd pulp (lees) was heat-sterilized at a temperature of 145 degrees Celsius for 5 seconds by an instantaneous heater that directly blows steam, and was cooled to a temperature of 5 degrees Celsius to obtain crude soymilk. Subsequently, appropriate amounts of additives such as sugar were added to the crude soymilk to obtain soymilk having its solid content adjusted to 10%.

When the soymilk thus obtained was measured for content of γ-aminobutyric acid and isoflavones by the method described below, the content of γ-aminobutyric acid in 125 mL of the soymilk was 15 mg, and that of isoflavones in 125 mL of the soymilk was 32 mg.

Analyses of γ-Aminobutyric Acid, Isoflavones, and Free Amino Acids

Analyses of γ-aminobutyric acid and free amino acids: After the soymilk thus obtained was mixed with 5% trichloroacetic acid, stirred, and centrifuged to remove proteins, the obtained supernatant was filtered, and the filtrate was analyzed with an automated amino acid analyzer. Contents of principal free amino acids are shown in Table 1.

Analysis of isoflavones: The obtained soymilk was homogenized in hydrated methanol at a mass ratio of methanol:water=8:2, extracted by heating under reflux for 1 hour twice, filtered, and the obtained filtrate was subjected to HPLC analysis for measuring isoflavones.

TABLE 1

| | | Comparative | |
|---|---|---|---|
| | Example 1 | Example 1 | Germinated/Non- |
| | (Germinated) | (Non-germinated) | germinated |
| Free Amino Acid | (mg/100 mL) | (mg/100 mL) | (%) |
| Thr | 2.30 | 0.31 | 742 |
| Ser | 2.67 | 0.45 | 589 |
| Met | 1.37 | 0.35 | 388 |
| Ile | 2.31 | 0.62 | 373 |
| Leu | 2.64 | 0.50 | 529 |
| Tyr | 2.44 | 0.40 | 612 |
| Phe | 2.80 | 0.68 | 411 |
| GABA | 11.68 | 0.49 | 2390 |
| Lys | 3.99 | 1.12 | 356 |
| His | 1.69 | 0.74 | 227 |

### Comparative Example 1

### Preparation of Ordinary Soymilk Using no Germinated Soybean

Using the same type of soybean as in Example 1, non-germinated soybeans were extracted by the usual hot water extraction method to obtain soymilk. That is, 300 kg of non-germinated soybeans (IOM, US) were thrashed, immersed in 1500 L of hot water at a temperature of 85 degrees Celsius, and crushed while stirring for about 30 minutes. The liquid obtained by separating soybean curd pulp was heated at a temperature of 145 degrees Celsius for 5 seconds by an instantaneous heater that directly blows steam, and cooled to a temperature of 5 degrees Celsius to obtain crude soymilk. Subsequently, appropriate amounts of additives such as sugar were added to the crude soymilk to obtain soymilk having the solid content adjusted to 10%. The obtained soymilk was analyzed for content of γ-aminobutyric acid and isoflavones by a method similar to that in Example 1. As a result, the content of γ-aminobutyric acid in 125 mL of the soymilk was 0 to 1 mg and that of isoflavones in 125 mL of the soymilk was 32 mg. The results of free amino acid analyses performed similarly to Example 1 are shown in Table 1.

Evaluation Test

Effects of soymilk of the present invention as a preventative and alleviative for menopausal disorders were evaluated in the test described below. In the following test example, the soymilk made from germinated soybeans of Example 1 and as a control, the ordinary soymilk prepared in Comparative Example 1 were used.

Test Method

One hundred and twenty women, from 40 to 59 years old, having menopausal disorders were stratified based on their age distribution, and randomly allotted to the following three groups A, B, and C (40 women per group). Soymilk was evaluated for its effects by giving it to women in each group as described below. In this case, the test was designed as a random comparative double blind test. Herein, “double blind test” refers to a test in which the treatment given to each subject is kept secret not only from the subject and the doctor conducting the therapeutical testing but also from the party requesting the testing and the staff of the doctor conducting the testing who participate in the treatment of the subject as well as the clinical evaluation.
(A) Experimental group: Each subject drinks 250 mL per day of the soymilk made from germinated soybeans according to Example 1 (daily dose of γ-aminobutyric acid: 30 mg, and that of isoflavones: 64 mg) continuously for 8 weeks;
(B) Placebo group: Each subject drinks 250 mL per day of the ordinary soymilk according to Comparative Example 1 (daily dose of γ-aminobutyric acid: 0 to 2 mg, and that of isoflavones: 64 mg) continuously for 8 weeks; and
(C) (Non-treated) control group: Each subject drinks no soymilk.

Evaluation was performed four times:

i) before the initiation of soymilk intake,

ii) 4 weeks after the initiation of soymilk intake,

iii) 8 weeks after the initiation of soymilk intake, and

iv) 4 weeks after the termination of soymilk intake. Using menopausal symptoms and quality of life as the evaluation indicators, the variations thereof in each group were analyzed by the following evaluation method and analysis method.

Evaluation Method and Data Analysis Method

Menopausal symptoms: They were evaluated using the simplified menopausal index according to Koyama. Specifically, a checklist concerning symptoms of menopausal disorders shown in the following Table 2 was prepared in advance, and the subjects scored degrees of their own symptoms according to the checklist based upon the criteria of “strong”, “medium”, and “weak”. These scores were given in figures, and the totals thereof were used as the menopausal indexes. The indexes at the initiation time of testing were compared to those at the respective evaluation times.

Symptoms were scored for the following 10 items and expressed in figures according to degrees thereof: (1) hot flushing and hot flashes; (2) hidrosis; (3) poor circulation at the waist and limbs; (4) shortness of breath and palpitations (the above-described items are vasomotor disorder-like symptoms); (5) dyscoimesis or light sleep; (6) being splenetic, and often irritable: (7) being fretful and depressed; (8) frequent headaches, vertigo, and nausea (the above-described items are psychoneuropathic disorder-like symptoms); (9) easily tiring; and (10) stiff neck, lumbago, and pains in the arms and legs (the above-described items are locomotor disorder symptoms). The case in which no symptoms are detected is scored as “none”; that in which symptoms are noticed, but do not significantly interfere with the subject's daily life is scored as “weak”; that in which symptoms are somewhat severe so as to be intolerable to the subject on occasions is scored as “medium”; and that in which symptoms are so severe as to disrupt the subject's daily life is scored as “strong”. These results were applied to the scores of respective items described in the questionnaire of menopausal disorders, and the totals thereof were used as the menopausal indexes.

Quality of life: The checklist of items (50 of them) concerning daily life discomfort is prepared in advance, and the subjects checked whether they have symptoms as listed in the checklist items, and added up the number of items checked to obtain a total score. Scores at the respective evaluation times were compared to those at the start of the testing.

Herein, 50 items concerning discomfort in daily life are as described in the following items (11) to (60).

(11) feeling suddenly lonely sometimes, (12) being irritated and restless, (13) feeling of secretly being spoken ill of by others and treated as a nuisance, (14) losing a sense of time and a sense of reality, (15) being hot-tempered or maudlin, (16) fussing over small details, (17) taking no pleasure in doing anything, (18) thinking about incessant troubles, (19) being bothered by troubles, (20) having lost the will to become enthusiastic, (21) having nothing to say to the family, (22) having no close friends any more, (23) having no association with relatives and neighbors any more, (24) having a person who stands in one's way, (25) having no desires to see anyone, (26) becoming very tired by speaking to others, (27) having no interest in the opposite sex, (28) being bothered by association with other people out of a sense of obligation, (29) not having good relationships with people near by, (30) having had troubles over the previous few months, (31) feeling of not having slept soundly, (32) having no appetite, (33) frequent constipation and diarrhea, (34) a frequent micturition and a sense of residual urine, (35) urinating sometimes due to only a slight movement, (36) unusual color in feces and urine, (37) having become too fat or too thin, (38) having headaches and being unable to think clearly, (39) feeling dizzy while standing and having vertigo sometimes, (40) having facial swelling, (41) easily having eye strain and viewed objects being distorted, (42) asking questions repeatedly sometimes, (43) having a pounding heart in spite of doing nothing, (44) being unable to stand up immediately, (45) often stumbling, (46) arms and legs being swollen or numb, (47) having stiff neck and lumbago, (48) always feeling sluggish, (49) having lost perseverance to do things, (50) having a disease does not clear up properly, (51) having a hard time in daily living, (52) being unable to work like everyone else, (53) daily life becoming a burden, (54) lacking the will to do something even though encouraged, (55) having neither dreams nor hopes for the future, and being gloomy about the future, (56) having lost aspirations, (57) being unable to worry about other people, (58) having little interest in changes in society, (59) not feeling enthusiasm for living, and (60) being unable to sympathize with other people.

TABLE 2

| Symptom | | Strong | Medium | Weak | None |
|---|---|---|---|---|---|
| Vasomotor | Hot Flashes | 10 | 6 | 3 | 0 |
| Disorder-like | Hidrosis | 10 | 6 | 3 | 0 |
| Symptoms | Poor Circulation | 14 | 9 | 5 | 0 |
| | in Waist and Limbs | | | | |
| | Short of Breath, | 12 | 8 | 4 | 0 |
| | Palpitations | | | | |
| Psychoneuro- | Dyscoimesis, | 14 | 9 | 5 | 0 |
| pathic | Light Sleep | | | | |
| Disorder-like | Being Splenetic | 12 | 8 | 4 | 0 |
| Symptoms | and Irritable | | | | |
| | Being Fretful | 7 | 5 | 3 | 0 |
| | and Depressed | | | | |
| | Headache, Vertigo, | 7 | 5 | 3 | 0 |
| | and Nausea | | | | |
| Locomotor | Easily Tiring | 7 | 4 | 2 | 0 |
| Disorder | Stiff Neck, | 7 | 5 | 3 | 0 |
| Symptoms | Lumbago, and | | | | |
| | Pains in Arms | | | | |
| | and Limbs | | | | |

Results

28 women of the experimental group as subjects enrolled in the analysis, 24 women of the placebo group, and 24 women of the control group were examined for menopausal symptoms and quality of life. Statistical analysis was performed by the corresponding t-test. The results were statistically analyzed for the presence of significant differences by comparing symptoms prior to the initiation of the test with those 4 weeks and those 8 weeks after the test initiation as well as those 4 weeks after the termination of the test. The results are shown in Table 3.

TABLE 3

| | | | | Comparison |
|---|---|---|---|---|
| | | Comparison | Comparison | before the Test |
| | | before and 4 | before and 8 | Initiation and 4 |
| | | Weeks after | Weeks after | Weeks after the |
| Menopausal | | the Test | the Test | Test |
| Disorder | Group | Initiation | Initiation | Termination |
| Vasomotor | (A) Experimental | ∘ | ∘ | ∘ |
| Disorder-like | Group | | | |
| Symptoms | (B) | — | ∘ | ∘ |
| | Placebo Group | | | |
| | (C) | — | — | — |
| | Control Group | | | |
| Psychoneuro- | (A) | ∘ | ∘ | ∘ |
| pathic Disorder- | Experimental | | | |
| like Symptoms | Group | | | |
| | (B) | — | — | ∘ |
| | Placebo Group | | | |
| | (C) | — | — | — |
| | Control Group | | | |
| Locomotor | (A) | — | ∘ | ∘ |
| Disorder | Experimental | | | |
| Symptoms | Group | | | |
| | (B) | — | ∘ | ∘ |
| | Placebo Group | | | |
| | (C) | — | — | — |
| | Control Group | | | |
| Quality of Life | (A) | — | ∘ | ∘ |
| | Experimental | | | |
| | Group | | | |
| | (B) | — | ∘ | — |
| | Placebo Group | | | |
| | (C) | — | — | — |
| | Control Group | | | |

∘: Significantly different (hazard rate 1%),
—: Not significantly different (hazard rate 1%).

As shown in Table 3, for the vasomotor disorder-like symptoms, a significant alleviation thereof was observed 4 weeks after the test initiation in the experimental group (A) (p<0.01). On the other hand, a significant alleviation of the symptoms was observed 8 weeks after the test initiation in the placebo group (B) (p<0.01), but not at both of the evaluation times in the (non-treated) control group (C).

As to the psychoneuropathic disorder-like symptoms, a significant alleviation thereof was observed 4 weeks after the test initiation in the experimental group (A) (p<0.01). On the other hand, in the placebo group (B), a significant alleviation of the symptoms was not observed even 8 weeks after the test initiation, but observed 4 weeks after the test termination (p<0.01). In the (non-treated) control group (C), no significant alleviation of the symptoms was observed at both of the evaluation times.

Furthermore, for the locomotor disorder symptoms, a significant alleviation of symptoms was observed 8 weeks after the test initiation in the experimental group (A) (p<0.01). On the other hand, in the placebo group (B), a significant alleviation of the symptoms was observed 8 weeks after the test initiation (p<0.01), but, in the (non-treated) control group (C), no significant alleviation of the symptoms was observed at both of the evaluation times.

From these results, it was shown that the vasomotor disorder-like symptoms are significantly alleviated in a shorter time in the experimental group (A) compared to the placebo group (B). Furthermore, it was shown that the psychoneuropathic disorder-like symptoms are significantly alleviated in the experimental group (A) in a much shorter time compared to the placebo group (B). On the other hand, for the locomotor disorder symptoms, there was no significant difference in the effects thereon between the experimental group (A) and placebo group (B). As to quality of life, it was shown that the experimental group (A) maintains long-term significant alleviation of symptoms even after the termination of soymilk intake, as compared to the placebo group (B).

### INDUSTRIAL APPLICABILITY

Soymilk of the present invention made from germinated soybean is capable of alleviating and preventing symptoms of menopausal disorders.

While preferred embodiments of the present invention have been described and illustrated above, it is to be understood that they are exemplary of the invention and are not to be considered to be limiting. Additions, omissions, substitutions, and other modifications can be made thereto without departing from the spirit or scope of the present invention. Accordingly, the invention is not to be considered to be limited by the foregoing description and is only limited by the scope of the appended claims.

What is claimed is:

1. Soymilk for alleviating or preventing vasomotor disorder-like symptoms and psychoneuropathic disorder-like symptoms associated with menopausal disorders, the soymilk comprising: processed germinated soybeans.

2. Soymilk made from germinated soybeans according to claim 1 wherein said vasomotor disorder-like symptoms are one or more symptoms selected from a group comprising heat, poor circulation, hot flashes, palpitation, tachycardia, and bradycardia, and wherein said psychoneuropathic disorder-like symptoms are one or more symptoms selected from a group comprising headache, vertigo, insomnia, susurrus aurium, phobias, oppressive feelings, and hypomnesia.

3. Soymilk made from germinated soybeans according to claim 1 wherein said soymilk is to be used for long-term improvement of quality of life (QOL) of people experiencing menopausal disorders.

4. Soymilk made from germinated soybeans according to claim 2 wherein said soymilk is to be used for long-term improvement of quality of life (QOL) of people experiencing menopausal disorders.

5. Soymilk made from germinated soybeans according to claim 1, wherein the soymilk contains γ-aminobutyric acid and isoflavones, and wherein the content of said γ-aminobutyric acid per 100 mL of the soymilk is not less than 8 mg, and that of said isoflavones per 100 mL of the soymilk is not less than 15 mg.

6. Soymilk made from germinated soybeans according to claim 2, wherein the soymilk contains γ-aminobutyric acid and isoflavones, and wherein the content of said γ-aminobutyric acid per 100 mL of the soymilk is not less than 8 mg, and that of said isoflavones per 100 mL of the soymilk is not less than 15 mg.

7. Soymilk made from germinated soybeans according to claim 3, wherein the soymilk contains γ-aminobutyric acid and isoflavones, and wherein the content of said γ-aminobutyric acid per 100 mL of the soymilk is not less than 8 mg, and that of said isoflavones per 100 mL of the soymilk is not less than 15 mg.

8. Soymilk made from germinated soybeans according to claim 4, wherein the soymilk contains γ-aminobutyric acid and isoflavones, and wherein the content of said γ-aminobutyric acid per 100 mL of the soymilk is not less than 8 mg, and that of said isoflavones per 100 mL of the soymilk is not less than 15 mg.

9. Soymilk made from germinated soybeans according to claim 5 wherein the soymilk contains serine, methionine, and phenylalanine, and has a solid content of not less than 8%, and wherein the content of said serine per 100 mL of the soymilk is not less than 2 mg, that of said methionine per 100 mL of the soymilk is not less than 1 mg, and that of said phenylalanine per 100 mL of the soymilk is not less than 2 mg.

10. Soymilk made from germinated soybeans according to claim 6 wherein the soymilk contains serine, methionine, and phenylalanine, and has a solid content of not less than 8%, and wherein the content of said serine per 100 mL of the soymilk is not less than 2 mg, that of said methionine per 100 mL of the soymilk is not less than 1 mg, and that of said phenylalanine per 100 mL of the soymilk is not less than 2 mg.

11. Soymilk made from germinated soybeans according to claim 7 wherein the soymilk contains serine, methionine, and phenylalanine, and has a solid content of not less than 8%, and wherein the content of said serine per 100 mL of the soymilk is not less than 2 mg, that of said methionine per 100 mL of the soymilk is not less than 1 mg, and that of said phenylalanine per 100 mL of the soymilk is not less than 2 mg.

12. Soymilk made from germinated soybeans according to claim 8 wherein the soymilk contains serine, methionine, and phenylalanine, and has a solid content of not less than 8%, and wherein the content of said serine per 100 mL of the soymilk is not less than 2 mg, that of said methionine per 100 mL of the soymilk is not less than 1 mg, and that of said phenylalanine per 100 mL of the soymilk is not less than 2 mg.
